(19) United States
(12) Patent Application Publication
(10) Pub. No.: US 2008/0146956 A1
(43) Pub. Date: Jun. 19, 2008

(54) Method based on a breath test for the detection of pathogen microorganisms

(76) Inventor: German A. Campuzano, Medellin (CO)

Correspondence Address: JOHN J. MARTINEZ MD. JD., 10 PALMER AVENUE, CROTON ON HUDSON, NY, 10520, US

(21) Appl. No.: 11/611,883

(22) Filed: Dec. 17, 2006

(51) Int. Cl. A61B 5/08

(52) U.S. Cl. 600/532

(57) ABSTRACT

The present invention provides a method based on urea breath tests for the detection and diagnostic of Helicobacter pylori in the gastroduodenal track, and a kit to carry out said method. The method comprises collecting a first basal breath sample from an individual or patient; giving to said individual a labeled carbon urea solution; in an immediate following novel and independent step, giving an adequate amount of water to the individual to clean his oral cavity from any residual urea; and after a time of not less than 5 minutes, collecting a second breath sample.

### FIELD OF THE INVENTION

The method of the present invention is related to the collection and analysis of breath samples for the detection of pathogen microorganisms. Specifically, the present invention provides a method based on the collection of breath samples, and a kit to carry out said collection, for the detection and diagnostic of Helicobacter pylori in the gastroduodenal track.

### BACKGROUND OF THE INVENTION

Helicobacter pylori has been mainly associated with gastroduodenal diseases, e.g., gastric or duodenal ulcer, dyspepsia, gastric non-Hodgkin's lymphomas, and gastric cancer. Moreover, the presence of H. pylori in the gastrointestinal track has also been associated with other diseases, e.g., chronic urticaria, iron-deficiency anemia, idiopathic thrombocytopenic purpura, etc. The list of diseases associated with H. pylori keeps growing.

Urea breath tests are reliable methods for the diagnosis of H. pylori. However the current gold standard protocols for urea breath test require the ingestion of citric acid solutions (see U.S. Pat. No. 6,171,811 B1 by De Bengoa Vallejo, A.). In addition said protocols could produce false negatives because of residual urea and urease activity from fixed microorganisms in the oral cavity. The ingestion of citric acid solutions in the urea breath test has been justified in part because the delay in gastric emptying caused by said solutions. However, the delay in gastric emptying has been shown not to be related to the ingestion of citric acid solutions (see Shiotani, A. S. et al., Aliment Pharmacol. Ther., 15:1763-1767, 2001) Furthermore, patients who ingest citric acid solutions during the urea breath test resist to take said solution due to worsening of gastroduodenal symptoms because the required suspension of anti-acids before the test, and the reasonable belief that the symptoms will get even worse if they drink something acid. Moreover, the ingestion of a load of citric acid can cause nauseous and vomit, specially, among children. Patients also complain about the high load and the bad taste of citric acid solutions. Furthermore, patients with allergy to citric acid can not have the current gold standard urea breath test.

The present invention provides a method based on urea breath tests that do not required citric acid solutions, and that also overcomes the presence of residual urea in the oral cavity. The method of the present invention provides a protocol that allows patients to go home after a few minutes without any side effects, or any worsening of gastric disease symptoms. In addition, the method of the present invention is, as reliable, or more reliable, than the current urea breath test protocols requiring the ingestion of citric acid solutions.

The present invention provides a method with an easy and reliable way to collect urea breath samples. The easy and reliable collection of urea breath samples will facilitate broad epidemiological studies to validate the possible association of H. Pylori with an increasing list of diseases.

### SUMMARY OF THE INVENTION

The present invention provides a method based on urea breath tests for the detection and diagnostic of Helicobacter pylori in the gastroduodenal track, and a kit to carry out said method.

The method comprises collecting a first basal breath sample from an individual or patient; giving to said individual a labeled carbon urea solution; in an immediate following novel independent step, giving an adequate amount of water to the individual to clean his oral cavity from any residual urea; and after a time of not less than 5 minutes, collecting a second breath sample.

The independent step of giving the individual an adequate amount of water has the dual purpose of cleaning the oral cavity of any residual labeled carbon urea and providing an optimal aqueous solution environment in the gastric cavity for a rapid diffusion and breakdown of the labeled carbon urea by the gastric H. pylori urease, therefore eliminating possible false positive results.

Objectives and additional advantages of the present invention will become more evident in the description of the figures, the detailed description of the invention and the claims.

### BRIEF DESCRIPTION OF THE DRAWINGS

FIG. 1 shows the kit and its components to carry out the method of the present invention.

### DETAILED DESCRIPTION OF THE INVENTION

The object of this invention is to provide a method based on breath tests for the detection of pathogen microorganisms comprising:
A. Collecting a first sample of an individual's basal breath;
B. Giving orally to the individual, immediately after collecting the first sample of basal breath, an urea solution with labeled carbon;
C. Giving orally to said individual, immediately after giving the urea solution with labeled carbon, an adequate amount of water;
D. Collecting a second breath sample, wherein the second breath sample is collected after a determined time from giving the adequate amount of water;
E. Measuring and analyzing the amount of labeled carbon present in the first breath sample and the second breath sample; and,

wherein the pathogen microorganism to be detected or diagnosed is Helicobacter pylori.

In one preferred aspect of the method of the present invention, the adequate amount of water is between 100 and 250 milliliters. However, in the most preferred embodiment, of said aspect of the method of the present invention, the adequate amount of water is 200 milliliters.

The novel independent step of giving the individual an adequate amount of just water, in a timely manner (right after giving said individual the labeled carbon urea solution), cleans the oral cavity from any residual labeled carbon urea, thus, eliminating the possibility of false positives because ureases of fixed microorganisms in the oral cavity. In addition, the adequate amount of water, once in the gastric cavity, provides an optimal intra-gastric aqueous solution and a homogeneous intra-gastric distribution of the carbon labeled carbon urea, and therefore a quick labeled carbon urea hydrolysis by the H. pylori urease.

In other preferred aspects of the method of the present invention, the urea solution with labeled carbon is between 15 and 50 milliliters; the urea with labeled carbon is between 15 and 50 milligrams; and the labeled carbon of the urea is carbon-13. The specific amounts, within the mentioned ranges, for both, the urea solution and the labeled carbon urea, are determined in accordance with the weight of the individual from who the breath samples are collected. Children would need amounts in the lower part of the ranges, while adults would need amounts in the upper part of the ranges.

The urea labeled with carbon-13 is preferably in the form of a powder, which is widely available commercially.

For the purpose of the present invention, individuals are defined as the patients or persons being tested for the presence of H. pylori in their gastrointestinal tracks.

In still another aspect of the method of the present invention, the second breath sample is collected more than 4 minutes and 59 seconds after giving the individual the adequate amount of water. In the most preferred embodiment of this aspect of the method of the present invention, the second breath sample is collected 10 minutes after giving the individual the adequate amount of water.

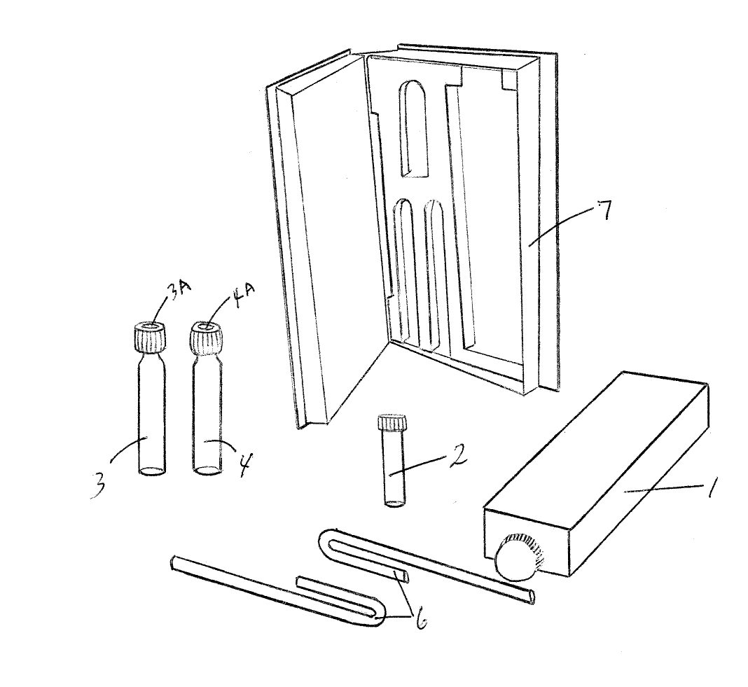

A second object of present invention is to provide a kit (FIG. 1) for the detection of pathogen microorganisms by mean of breath tests, wherein the kit comprises:
a. A container with water (1)(FIG. 1);
b. A container with labeled carbon urea (2);
c. A first receptacle (3) to collect a first breath sample at the beginning of the breath test and before the ingestion of labeled carbon urea;
d. A second receptacle (4) to collect a second breath sample at the end of the breath test and after the ingestion of an adequate amount of water; and,

wherein the first receptacle and the second receptacle have a cap with a mechanism (3A and 4A) that allows the introduction of a needle shaped sensor, and wherein the pathogen microorganism to be detected or diagnosed is H. pylori.

The first and second samples collected, in the properly differentiated first receptacle and second receptacle respectively, are analyzed to determine the amounts of carbon-13 by gas chromatography and gas spectrometry.

In one aspect of the kit of the present invention, the container with labeled carbon urea contains between 15 and 50 milligrams of labeled carbon urea, and wherein the labeled carbon urea is dissolved in water, and wherein the amount of water to dissolve the labeled carbon urea is between 15 and 50 milliliters, and wherein the labeled carbon of the urea is carbon-13. In the most preferred embodiment, the amount of labeled carbon urea is 50 milligrams.

In another preferred aspect of the kit of the present invention, the container with water contains between 200 and 250 milliliters of water. However, in the most preferred embodiment, of said aspect of the kit of the present invention, the adequate amount of water is 200 milliliters.

In still another aspect of the kit of the present invention, the kit comprises a pair of means to collect the first breath sample and the second breath sample into the first receptacle and the second receptacle. In a preferred embodiment of this aspect of the kit of the present invention, the means to collect the first breath sample and the second breath sample into the first receptacle and the second receptacle are a pair of straws (6).

For the purpose of the present invention, receptacle is defined as any container which can be air tight sealed. In a preferred embodiment of the invention the receptacles are tubular containers.

In one more aspect of the kit of the present invention, a cartridge (7) contains the container with water, the container with labeled carbon urea, the first receptacle, the second receptacle, and the pair of straws.

While the description presents the preferred embodiments of the present invention, additional changes can be made in the form and disposition of the parts without distancing from the basic ideas and principles comprised in the claims.

### EXAMPLE

### Material and Methods

Patients: The population of the study consisted of 70 healthy volunteers who were gastrointestinally asymptomatic. In accordance with the Helsinki declaration and the Health Ministry of Colombia Resolution 8430 of 1993, the research is classified as research without biological, physiological, psychological or social risks.

Labeled Carbon Urea Breath Test:

PROTOCOL 1: After fasting for at least 8 hours, a first basal breath sample was collected (t₀); immediately after, the patient was given orally 50 mg ¹³C labeled urea dissolved in 15 ml of water in a single drink; immediately after, the patient was given orally 200 ml of water; then, after 10 (t₁₀), 20 (t₂₀) and 30 (t₃₀) minutes, breath samples were collected.

PROTOCOL 2: After fasting for at least 8 hours, a first basal breath sample was collected (to); immediately after, the patient was given orally 50 mg ¹³C labeled urea dissolved in 15 ml of water in a single drink; immediately after the patient was given 200 ml of water with 4.2 g of dehydrated citric acid; then, after 10 (t₁₀), 20 (t₂₀) and 30 (t₃₀) minutes, breath samples were collected.

STANDARD PROTOCOL OF REFERENCE: The European protocol standardized for the Colombian environment was used as the reference protocol which has been broadly validated with sensitivity and specificity closed to 100%. It was considered not ethical to perform invasive tests, e.g. biopsy, culture and endoscopy. The reference protocol was performed as follows: After fasting for at least 8 hours, the patient was given 4.2 of citric acid dissolved in 100 ml of water; ten minutes after, a duplicate basal breath sample was collected (to); immediately after, the patient was given 100 mg of ¹³C labeled urea dissolved in 30 ml of water; then, after 30 minutes, a duplicate post-urea breath sample was collected.

Definition of Infection by H. pylori: The infection status by H. Pylori was defined by means of a ¹³C labeled urea breath test in accordance with the reference protocol with commercial kits (TAU-KIT®, Isomed S. L., Madrid, Spain). The samples analysis was performed in the Laboratorio Clinico Hematológico S. A., Medellin, Colombia, using a spectrometer (ABCA, Europa Scientific, Cheshire, United Kingdom), in accordance with internationally validated criteria for this test: a positive result was a value for the ¹³C labeled urea breath test equal or superior to 2.5 ¹³CO₂ deltas (δ¹³CO₂). The results were expressed as deltas over the base line (DOB). The cutoff points for each one of the protocols varied from 0.5 to 5.5 in different time intervals (10, 20 and 30 minutes). The effectiveness of the each protocol was evaluated with the ROC curves.

Statistical Analysis: In the descriptive analysis, absolute and relative distributions were used for qualitative variables. Resume indicators were used for quantitative variables. Normality criteria for the data were established with the Shapiro-Wilk test, and based on this, the t-student test, and the Wilcoxon sign range test, were applied to establish the difference among independent medians. The independence X² test was used to analyze associations between qualitative variables. A value of p<0.05 was considered statistically significant. In addition, sensitivity, specificity, positive predictive value, negative predictive value, the validity index, the Youden index, the verisimilitude reason for positive results (RV+), and the verisimilitude reason for negative results (RV−), were calculated. Processing and analysis of obtained data were done with the SPSS ((Statistical Product for Service Solutions) Version 12.0, and EPIDATE Version 3.0 programs.

Results: In the study, 70 asymptomatic individuals were included (24 males and 46 females), wherein said individuals have an average age of 39.63 (SD±12.58) years for males and 34.33 (SD±10.17) years for females; with a 95% confidence. There were no significant differences between the average ages for males and females (t-Student=1.905; p=0.061).

Using the Reference Protocol, 46 (65.7%) individuals were positive for H. pylori, and 24 (34.3%) individuals were negative for H. pylori. By sex, 17 (70.8%) males and 29 (63%) females were positive for H. pylori. There was no significant statistical difference between males and females (X²=0.425; p=0.515). For Protocols I and II, results can be observed in Table 1.

TABLE 1
¹³C labeled urea breath test. Validity index (Índice de validez), Youden Index (índice de Youden).
RV+ and RV−, at different cutoff points (10, 20 y 30 minutes), for protocols I y II.
Laboratorio Clinico Hematológico S.A, Medellín, Colombia 2006.

| | | Índice de Validez | | Índice Youden | | RV+ | | RV− | |
|---|---|---|---|---|---|---|---|---|---|
| Minuto | Valor δ P.C. | Protocolo I | Protocolo II | Protocolo I | Protocolo II | Protocolo I | Protocolo II | Protocolo I | Protocolo II |
| 10 | 0.5 | 81.43 | 85.71 | 0.46 | 0.60 | 1.85 | 2.61 | * | 0.03 |
| | 1 | 94.29 | 91.43 | 0.83 | 0.77 | 6.00 | 4.70 | * | 0.03 |
| | 1.5 | 98.57 | 97.14 | 0.96 | 0.94 | 24.00 | 23.48 | * | 0.02 |
| | 2 | 100.00 | 98.57 | 1.00 | 0.98 | ** | ** | * | 0.02 |
| | 2.5 | 100.00 | 97.14 | 1.00 | 0.96 | ** | ** | * | 0.04 |
| | 3 | 98.57 | 97.14 | 0.98 | 0.96 | ** | ** | 0.02 | 0.04 |
| | 3.5 | 98.57 | 97.14 | 0.98 | 0.96 | ** | ** | 0.02 | 0.04 |
| | 4 | 97.14 | 97.14 | 0.96 | 0.96 | ** | ** | 0.04 | 0.04 |
| | 4.5 | 95.71 | 97.14 | 0.93 | 0.96 | ** | ** | 0.07 | 0.04 |
| | 5 | 91.43 | 97.14 | 0.87 | 0.96 | ** | ** | 0.13 | 0.04 |
| | 5.5 | 90.00 | 97.14 | 0.85 | 0.96 | ** | ** | 0.15 | 0.04 |
| 20 | 0.5 | 88.57 | 85.71 | 0.67 | 0.60 | 3.00 | 2.61 | * | 0.03 |
| | 1 | 98.57 | 90.00 | 0.96 | 0.73 | 24.00 | 3.91 | * | 0.03 |
| | 1.5 | 100.00 | 92.86 | 1.00 | 0.81 | ** | 5.87 | * | 0.03 |
| | 2 | 100.00 | 98.57 | 1.00 | 0.98 | ** | ** | * | 0.02 |
| | 2.5 | 100.00 | 97.14 | 1.00 | 0.96 | ** | ** | * | 0.04 |
| | 3 | 98.57 | 97.14 | 0.98 | 0.96 | ** | ** | 0.02 | 0.04 |
| | 3.5 | 97.14 | 97.14 | 0.96 | 0.96 | ** | ** | 0.04 | 0.04 |
| | 4 | 92.86 | 97.14 | 0.89 | 0.96 | ** | ** | 0.11 | 0.04 |
| | 4.5 | 90.00 | 97.14 | 0.85 | 0.96 | ** | ** | 0.15 | 0.04 |
| | 5 | 88.57 | 97.14 | 0.83 | 0.96 | ** | ** | 0.17 | 0.04 |
| | 5.5 | 88.57 | 95.71 | 0.83 | 0.93 | ** | ** | 0.17 | 0.07 |
| 30 | 0.5 | 84.29 | 80.00 | 0.54 | 0.44 | 2.18 | 1.81 | * | 0.05 |
| | 1 | 97.14 | 88.57 | 0.92 | 0.69 | 12.00 | 3.35 | * | 0.03 |
| | 1.5 | 100.00 | 92.86 | 1.00 | 0.81 | ** | 5.87 | * | 0.03 |
| | 2 | 97.14 | 95.71 | 0.96 | 0.89 | ** | 11.74 | 0.04 | 0.02 |
| | 2.5 | 94.29 | 98.57 | 0.91 | 0.98 | ** | ** | 0.09 | 0.02 |
| | 3 | 90.00 | 97.14 | 0.85 | 0.96 | ** | ** | 0.15 | 0.04 |
| | 3.5 | 88.57 | 97.14 | 0.83 | 0.96 | ** | ** | 0.17 | 0.04 |
| | 4 | 85.71 | 97.14 | 0.78 | 0.96 | ** | ** | 0.22 | 0.04 |
| | 4.5 | 85.71 | 97.14 | 0.78 | 0.96 | ** | ** | 0.22 | 0.04 |
| | 5 | 84.29 | 97.14 | 0.76 | 0.96 | ** | ** | 0.24 | 0.04 |
| | 5.5 | 81.43 | 95.71 | 0.72 | 0.93 | ** | ** | 0.28 | 0.07 |

* ≈ 0
** φ +
PREVALENCE: 65.71
Minute (Minuto),
Value δ P.C. (Valor δ P.C.),
Protocol (Protocolo).

The best performing cutoff points, for the Protocol I, is found at 10 minutes, 2.0 and 2.5 deltas (100% for both); at 20 minutes, 1.5, 2.0, and 2.5 deltas (100% for all three); at 30 minutes, 1.5 deltas (100%); and for Protocol II, at ten minutes, 2.0 deltas (98.57%); at 20 minutes, 2.0 deltas (98.57%), at 30 minutes, 2.5 deltas (98.57%). An evaluation of the ¹³C labeled urea breath test efficiency, according to the Youden Index, shows that the Protocol I was a perfect diagnostic test at the best performance points. The efficacy can be seen by looking the RV+ which indicates that Protocols I and II have a high probability of classifying H. Pylori infected individuals as positive, while the RV− indicates that Protocols I and II have a probability close to zero (0) of classifying H. Pylori non-infected individuals as positive.

Table 2 shows that at the best performing cutoff points, 10, 20 and 30 minutes, the diagnostic ¹³C labeled urea breath test, for Protocol, I has a sensitivity of 100%. In contrast, at the best performing cutoff points, 10, 20 and 30 minutes, the diagnostic ¹³C labeled urea breath test, for Protocol II, has a sensitivity of 97.83%. The specificity for both, Protocol I and Protocol II, was 100%.

With a confidence of 95%, there were no statistical significant differences between Protocol I and Protocol II, at 10 minutes (X²=0.9857; p=0.3208), at 20 minutes (X²=0.9699; p=0.3247), and at 30 minutes (X²=0.9857; p=0.3208). The results for both protocols at 10 and 30 minutes were similar.

TABLE 2
¹³C labeled urea breath test. Sensitivity (Sensibilidad), Specificity (Especificidad), Positive Predictive Value
(Valor Predictivo +), and Negative Predictive Value (Valor Predictivo −) for Protocols I and II, at different cutoff
points, 10, 20 y 30 minutes. Laboratorio Clínico Hematológico S.A, Medellín, Colombia 2006.

| | | Sensibilidad | | Especificidad | | Valor Predictivo+ | | Valor Predictivo− | |
|---|---|---|---|---|---|---|---|---|---|
| Minuto | Valor δ P.C. | Protocolo I | Protocolo II | Protocolo I | Protocolo II | Protocolo I | Protocolo II | Protocolo I | Protocolo II |
| 10 | 0.5 | 100.00 | 97.83 | 45.83 | 62.50 | 77.97 | 83.33 | 100.00 | 93.75 |
| | 1 | 100.00 | 97.83 | 83.33 | 79.17 | 92.00 | 90.00 | 100.00 | 95.00 |
| | 1.5 | 100.00 | 97.83 | 95.83 | 95.83 | 97.87 | 97.83 | 100.00 | 95.83 |
| | 2 | 100.00 | 97.83 | 100.00 | 100.00 | 100.00 | 100.00 | 100.00 | 96.00 |
| | 2.5 | 100.00 | 95.65 | 100.00 | 100.00 | 100.00 | 100.00 | 100.00 | 92.31 |
| | 3 | 97.83 | 95.65 | 100.00 | 100.00 | 100.00 | 100.00 | 96.00 | 92.31 |
| | 3.5 | 97.83 | 95.65 | 100.00 | 100.00 | 100.00 | 100.00 | 96.00 | 92.31 |
| | 4 | 95.65 | 95.65 | 100.00 | 100.00 | 100.00 | 100.00 | 92.31 | 92.31 |
| | 4.5 | 93.48 | 95.65 | 100.00 | 100.00 | 100.00 | 100.00 | 88.89 | 92.31 |
| | 5 | 86.96 | 95.65 | 100.00 | 100.00 | 100.00 | 100.00 | 80.00 | 92.31 |
| | 5.5 | 84.78 | 95.65 | 100.00 | 100.00 | 100.00 | 100.00 | 77.42 | 92.31 |
| 20 | 0.5 | 100.00 | 97.83 | 66.67 | 62.50 | 85.19 | 83.33 | 100.00 | 93.75 |
| | 1 | 100.00 | 97.83 | 95.83 | 75.00 | 97.87 | 88.24 | 100.00 | 94.74 |
| | 1.5 | 100.00 | 97.83 | 100.00 | 83.33 | 100.00 | 91.84 | 100.00 | 95.24 |
| | 2 | 100.00 | 97.83 | 100.00 | 100.00 | 100.00 | 100.00 | 100.00 | 96.00 |
| | 2.5 | 100.00 | 95.65 | 100.00 | 100.00 | 100.00 | 100.00 | 100.00 | 92.31 |
| | 3 | 97.83 | 95.65 | 100.00 | 100.00 | 100.00 | 100.00 | 96.00 | 92.31 |
| | 3.5 | 95.65 | 95.65 | 100.00 | 100.00 | 100.00 | 100.00 | 92.31 | 92.31 |
| | 4 | 89.13 | 95.65 | 100.00 | 100.00 | 100.00 | 100.00 | 82.76 | 92.31 |
| | 4.5 | 84.78 | 95.65 | 100.00 | 100.00 | 100.00 | 100.00 | 77.42 | 92.31 |
| | 5 | 82.61 | 95.65 | 100.00 | 100.00 | 100.00 | 100.00 | 75.00 | 92.31 |
| | 5.5 | 82.61 | 93.48 | 100.00 | 100.00 | 100.00 | 100.00 | 75.00 | 88.89 |
| 30 | 0.5 | 100.00 | 97.83 | 54.17 | 45.83 | 80.70 | 77.59 | 100.00 | 91.67 |
| | 1 | 100.00 | 97.83 | 91.67 | 70.83 | 95.83 | 86.54 | 100.00 | 94.44 |
| | 1.5 | 100.00 | 97.83 | 100.00 | 83.33 | 100.00 | 91.84 | 100.00 | 95.24 |
| | 2 | 95.65 | 97.83 | 100.00 | 91.67 | 100.00 | 95.74 | 92.31 | 95.65 |
| | 2.5 | 91.30 | 97.83 | 100.00 | 100.00 | 100.00 | 100.00 | 85.71 | 96.00 |
| | 3 | 84.78 | 95.65 | 100.00 | 100.00 | 100.00 | 100.00 | 77.42 | 92.31 |
| | 3.5 | 82.61 | 95.65 | 100.00 | 100.00 | 100.00 | 100.00 | 75.00 | 92.31 |
| | 4 | 78.26 | 95.65 | 100.00 | 100.00 | 100.00 | 100.00 | 70.59 | 92.31 |
| | 4.5 | 78.26 | 95.65 | 100.00 | 100.00 | 100.00 | 100.00 | 70.59 | 92.31 |
| | 5 | 76.09 | 95.65 | 100.00 | 100.00 | 100.00 | 100.00 | 68.57 | 92.31 |
| | 5.5 | 71.74 | 93.48 | 100.00 | 100.00 | 100.00 | 100.00 | 64.86 | 88.89 |

Minute (Minuto),
Value δ P.C. (Valor δ P.C.),
Protocol (Protocolo).

According to the ROC areas (Graphic 1), the ¹³C labeled urea breath test under Protocol I is a perfect test, with both, sensitivity and specificity equal to 1.

Table 3 and Graphic 2, show the characterization of cutoff point values [γ¹³CO₂] at 10, 20, 30 minutes for H. pylori positive and negative individuals for Protocols I and II.

TABLE 3
Distribution of values δ¹³CO₂ of positive y negative individuals for Helicobacter pylori,
for Protocols (Protocolos) I and II at 10, 20 y 30 minutes (minutos). Laboratorio Clínico
Hematológico S.A, Medellín, 2006.

| | | | | | |
|---|---|---|---|---|---|
| Protocolo I | Protocolo I | Protocolo I | Protocolo II | Protocolo II | Protocolo II |
| 10 Minutos | 20 Minutos | 30 Minutos | 10 Minutos | 20 Minutos | 30 Minutos |

POSITIVOS H. pylori

| | | | | | | |
|---|---|---|---|---|---|---|
| Media | 17.38 | 18.25 | 15.44 | 17.69 | 22.32 | 22.08 |
| Mediana | 13.64 | 12.63 | 10.98 | 12.02 | 17.07 | 17.50 |
| Desviación Estándar | 14.47 | 22.80 | 17.75 | 12.68 | 15.01 | 13.00 |
| Mínimo | 2.84 | 2.63 | 1.60 | −0.11 | 0.21 | 0.19 |
| Máximo | 66.08 | 149.80 | 107.90 | 52.45 | 59.02 | 54.79 |
| Cuartil Inferior | 6.59 | 6.20 | 5.13 | 8.54 | 11.11 | 13.26 |
| Cuartil Superior | 21.87 | 22.35 | 23.30 | 25.22 | 32.35 | 30.53 |

NEGATIVOS H. pylori

| | | | | | | |
|---|---|---|---|---|---|---|
| Media | 0.32 | 0.11 | 0.21 | 0.33 | 0.45 | 0.62 |
| Mediana | 0.51 | 0.28 | 0.38 | 0.32 | 0.34 | 0.59 |
| Desviación Estándar | 0.91 | 0.77 | 0.84 | 0.80 | 0.86 | 0.91 |
| Mínimo | −2.17 | −2.03 | −2.22 | −1.97 | −1.94 | −1.89 |
| Máximo | 1.76 | 1.18 | 1.07 | 1.98 | 1.88 | 2.13 |
| Cuartil Inferior | −0.29 | −0.19 | −0.01 | −0.15 | 0.07 | 0.20 |
| Cuartil Superior | 0.95 | 0.73 | 0.72 | 0.88 | 1.16 | 1.26 |

Positive (Positivo),
Negative (Negativos),
Media (Media),
Median (Mediana),
Standard Deviation (Desviación Estandar),
Minimun (Minimo),
Maximum (Maximo),
Inferior Quartile (cuartel inferior),
Superior Quartile (Cuartel Superior)

Table 3 and Graphic 2 also show that for the ¹³C labeled urea breath tests—Protocol I, the δ¹³CO₂ median for H. Pylori infected individuals, at 10 minutes, was 13.64, while the δ¹³CO₂ median for the Protocol II was 12.02. There was no statistical significant difference between these two values (Wilcoxon, p=0.121). In contrast, at 20 and 30 minutes, there were statistical significant differences (Wilcoxon, p=0.006, and p 0.001 respectively).

In addition, Table 3 and Graphic 2 show that for the ¹³C labeled urea breath tests—Protocol I, the δ¹³CO₂ median for non-infected individuals, at 10 minutes, was 0.51, while the δ¹³CO₂ median for the Protocol II was 0.32. There were no statistical significant differences for these values at 10, 20 and 30 minutes (Wilcoxon, p=0.710, p=0.440 and p=0.346 respectively)

Discussion: The results suggest that the use of citric acid, as part of the ¹³C labeled urea breath test, may not be necessary. The sensitivity and specificity of Protocol I also suggest that the novel independent step of giving an adequate amount of water to the individual being tested is an appropriate alternative, instead of capsules and endoscopic intra-gastric instillation, to avoid the contact of residual ¹³C labeled urea with other possible urease producing bacteria fixed in the oral cavity. The results for protocol I at 10 minutes, also suggest that the novel independent step of giving an adequate amount of water to the individual being tested may provide optimal conditions for a quick ¹³C labeled urea breakdown by the H. pylori urease in the gastric cavity. The results of this study also suggest that a 50 mg dose of ¹³C labeled urea is adequate for a test with optimal conditions regarding sensitivity, specificity, positive predictive value and negative predictive value, as it is shown in Table 2.

Eliminating citric acid from the ¹³C labeled urea breath test results in more tolerable test, since the aqueous solution with ¹³C labeled urea is colorless, odorless, tasteless and innocuous.

In conclusion, Protocol I provides a ¹³C labeled urea breath test that is well tolerated, easier to do, of less duration, and wherein the amount of ¹³C labeled urea is reduced. The collection of samples for the test of Protocol I can be performed remotely and the collected samples can be sent by mail, since the collected samples can be stored at room temperature for months without negatively affecting the ¹³C content in the individual's breath. Protocol I provides a test that can be used massively for epidemiological studies and broad eradication of H. Pylori.

1. A method based on breath tests for the detection of pathogen microorganisms comprising:
F. Collecting a first sample of an individual's basal breath;
G. Giving orally to the individual, immediately after collecting the first sample of basal breath, an urea solution with labeled carbon;
H. Giving orally to said individual, immediately after giving the urea solution with labeled carbon, an adequate amount of water;
I. Collecting a second breath sample, wherein the second breath sample is collected after a determined time from giving the water; and,
J. Measuring and analyzing the amount of labeled carbon present in the first breath sample and the second breath sample.

2. The method of claim 1, wherein the adequate amount of water is between 100 and 250 milliliters.

3. The method of claim 1, wherein the urea solution with labeled carbon is between 15 and 50 milliliters, and wherein the urea with labeled carbon is between 15 and 50 milligrams, and wherein the labeled carbon of the urea is carbon-13.

4. The method of claim 1, wherein the second breath sample is collected more than 4 minutes and 59 seconds after giving the individual the adequate amount of water.

5. The method of claim 1, wherein the pathogen microorganism is Helicobacter pylori.

6. A kit for the detection of pathogen microorganisms by mean of breath tests, wherein the kit comprises:
e. A container with water;
f. A container with labeled carbon urea;
g. A first receptacle to collect a first breath sample at the beginning of the breath test and before the ingestion of labeled carbon urea;
h. A second receptacle to collect a second breath sample at the end of the breath test and after the ingestion of an adequate amount of water; and,
wherein the first receptacle and the second receptacle have a cap with a mechanism that allows the introduction of a needle shaped sensor.

7. The kit of claim 6, wherein the container with labeled carbon urea contains between 15 and 50 milligrams of labeled carbon urea, and wherein the labeled carbon urea is dissolved in water, and wherein the amount of water to dissolve the labeled carbon urea is between 15 and 50 milliliters.

8. The kit of claim 6, wherein the labeled carbon urea is labeled with carbon-13.

9. The kit of claim 6, wherein the container with water contains between 200 and 250 milliliters of water.

10. The kit of claim 6, wherein said kit comprises a pair of means to collect the first breath sample and the second breath sample into the first receptacle and the second receptacle respectively.

11. The kit of claim 10, wherein said means to collect the first breath sample and the second breath sample are a pair of straws.

12. The kit of claim 11, wherein the container with water, the container with labeled carbon urea, the first receptacle, the second receptacle, and the pair of straws, are contained within a cartridge.

13. The kit of claim 10, wherein the pathogen microorganism is Helicobacter pylori.
